(19) United States
(12) Patent Application Publication
(10) Pub. No.: US 2007/0184130 A1
(43) Pub. Date: Aug. 9, 2007

(54) Formulation and method of use for animal ear treatment and homeopathic animal ear treatment

(76) Inventor: Michael Glenn Carrigan, Highland Village, TX (US)

Correspondence Address: DOCKET CLERK, P.O. DRAWER 800889, DALLAS, TX, 75380, US

(21) Appl. No.: 11/702,394

(22) Filed: Feb. 3, 2007

Related U.S. Application Data
(60) Provisional application No. 60/765,461, filed on Feb. 3, 2006.
(60) Provisional application No. 60/811,401, filed on Jun. 6, 2006.

(51) Int. Cl. A61K 36/82; A61K 31/695

(52) U.S. Cl. 424/729; 424/725; 514/63

(57) ABSTRACT

An animal ear treatment solution is formulated with an organosilane polymer antimicrobial in a concentration of approximately 0.25% to approximately 3.0% by weight, a surfactant or homeopathic active ingredient, and an optional holistic antiseptic such as green tea oil or eucalyptus oil. The solution is formulated to have a pH of about 10 but less than 12. Weekly application of the solution within an animal's ear canal allows the organosilane polymer antimicrobial to attach to tissue within the ear canal, forming a mechanical barrier against growth of bacteria on such tissue. The holistic antiseptic, if present within the solution, acts to destroy bacteria within the ear canal.

This application claims priority to co-pending U.S. Provisional Patent Application No. 60/765,461 filed Feb. 3, 2006 entitled FORMULATION AND METHOD OF USE FOR ANIMAL EAR TREATMENT and co-pending U.S. Provisional Patent Application No. 60/811,401 filed Jun. 6, 2006 entitled FORMULATION AND METHOD OF USE FOR HOMEOPATHIC ANIMAL EAR TREATMENT. The content of the above-identified applications is incorporated herein by reference.

### TECHNICAL FIELD

The present disclosure is directed, in general, to animal ear treatments and, more specifically, to a homeopathic treatment containing an active ingredient that optionally also functions as a preservative, and a bonding agent, and a holistic antiseptic.

### BACKGROUND

Many animals, particularly domestic canines, are susceptible to ear infections due to a vertical drop in portions of the ear canal between an innermost region and an outermost region. Debris entering the ear canal of such animals must work its way upward rather than simply straight out. Foreign bodies within the ear canal are a significant source of ear infections in such animals.

Irritants within the ear canal may also cause overproduction of wax in many breeds of animals, representing another source of ear disease. Chronic ear problems are not uncommon in some breeds, with as many as one-quarter of all domestic canines exhibiting such recurring ear problems. Discomfort is manifested by the animal through shaking of the head and ears (which may result in blood vessels in the ear rupturing and bleeding to create clots within the ear), rubbing ears on structures (floor/ground, furniture or building structures) and scratching of the ears.

There is, therefore, a need in the art for an animal ear treatment providing effective protection against ear infections and other ear diseases within the treated animal.

### SUMMARY

To address the above-discussed deficiencies of the prior art, a formulation for use as an animal ear treatment includes a solution with an organosilane polymer antimicrobial in a concentration of approximately 0.25% to approximately 3.0% by weight, a surfactant, and an optional holistic antiseptic such as green tea oil or eucalyptus oil. The solution is formulated to have a pH of about 6 but less than 12, preferably about 10.

An alternative formulation includes a solution with: an organosilane polymer anti-microbial in a concentration of approximately 0.25% to approximately 3.0% by weight; a surfactant; homeopathic active ingredient(s) of up to 2.00% by volume each of calendula officinalis tinc in 45% alcohol; mercurius solubilis in 20% alcohol, and rhus toxicodendron in 20% alcohol; and an optional holistic antiseptic such as eucalyptus oil or tea tree oil.

Weekly application of the solution within an animal's ear canal allows the organosilane polymer antimicrobial to attach to tissue within the ear canal, forming a mechanical barrier against growth of bacteria on such tissue. The holistic antiseptic, if present within the solution, acts to destroy bacteria within the ear canal.

The foregoing has outlined rather broadly the features and technical advantages of the present disclosure so that those skilled in the art may better understand the detailed description of the subject matter that follows. Additional features and advantages of the disclosure will be described hereinafter that form the subject of the claims. Those skilled in the art will appreciate that they may readily use the conception and the specific embodiment disclosed as a basis for modifying or designing other formulations for carrying out the same purposes of the present disclosure. Those skilled in the art will also realize that such equivalent constructions do not depart from the spirit and scope of the disclosure in its broadest form.

Before undertaking the DETAILED DESCRIPTION below, it may be advantageous to set forth definitions of certain words or phrases used throughout this patent document: the terms “include” and “comprise,” as well as derivatives thereof, mean inclusion without limitation; the term “or” is inclusive, meaning and/or; and the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. Definitions for certain words and phrases are provided throughout this patent document, and those of ordinary skill in the art will understand that such definitions apply in many, if not most, instances to prior as well as future uses of such defined words and phrases.

### DETAILED DESCRIPTION

The various embodiments used to describe the principles of the present disclosure in this patent document are by way of illustration only and should not be construed in any way to limit the scope of the disclosure. Those skilled in the art will understand that the principles of the present disclosure may be implemented in or with any suitably formulated treatment.

One formulation of the animal ear treatment in the present disclosure includes an active ingredient, a bonding agent, and a preservative. One suitable active ingredient is the AEM 5700 antimicrobial solution (colloquially referred to as “5700”) available from Aegis Environments of Midland, Mich., marketed under the brand name “Aegis Microbe Shield.” This solution contains approximately 84% octadecylaminodimethyltrihydroxysilyl-propyl ammonium chloride and approximately 16% chloro-propyltrihydroxysilane. Suitably, the antimicrobial active ingredient exhibits no untoward effects for eye or skin exposure. Advantageously, the antimicrobial active agent also acts as a preservative. Thus, when an optional holistic antiseptic is included, as described below, the formulation may be accurately labeled as an herbal remedy, with the antimicrobial active agent functioning as a preservative.

The animal ear treatment formulation may include up to about 3% of an antimicrobial active ingredient or about 0.25% to about 3% of an antimicrobial active ingredient. Alternatively, the animal ear treatment formulation may include about 1% to about 1.25% of the antimicrobial active ingredient. In accordance with certain embodiments, the animal ear treatment formulation may include up to about 3% of an organosilane polymer antimicrobial ingredient such as, for example, a blend of octadecylaminodimethyltrihydroxysilyl-propyl ammonium chloride and chloropropyltrihydroxysilane. Suitably, the animal ear treatment formulation may include about 0.25% to about 3% of the organosilane polymer antimicrobial ingredient or about 1% to about 1.25%.

A holistic antiseptic such as, for example, an essential oil having antiseptic properties, may be added to the ear treatment as desired. Suitable essential oils having antiseptic properties which may be included in the ear treatment include, but are not limited to, tea tree oil, eucalyptus oil or combinations thereof. For example, the ear treatment formulation may include up to about 3% of a holistic antiseptic or about 1% to about 2% of a holistic antiseptic.

To formulate approximately ten gallons of the ear treatment, or enough to fill about 160, 8 ounce (oz) bottles, water is poured into a container and the 5700 (and antiseptic, if any) is slowly added to the water over a period of about three minutes, while constantly stirring the solution. A surfactant or wetting agent, such as a nonionic soap that remains in solution, is added as the bonding agent over a period of approximately two minutes while constantly stirring the mixture, and then the solution is mixed for about another two minutes for thorough mixing.

While stirring and constantly monitoring the concentration of hydrogen ions (pH), a solution comprising 50 grams per liter (g/L) of sodium hydroxide (NaOH) is added to the mixture very slowly until a pH at or near 10 is achieved. Errors on the high side of the pH scale are better, such that a pH of slightly over 10 is acceptable and preferable to a pH of slightly under 10. However, a pH greater than about 12 can render the solution useless. For that reason, adding the sodium hydroxide solution too fast and/or with insufficiently thorough mixing (uniformity within the mixture) can destroy the solution. After a stable acceptable pH is achieved, stirring of the solution should continue for two additional minutes.

TABLE I below details details one example of an animal ear treatment formulation according to this disclosure:

TABLE I

| | Specific | Percent | Volume | | Mass |
|---|---|---|---|---|---|
| Ingredient | Gravity | by volume | L | mL | (g) |
| Water | 1 | 97.94% | 37.0731 | 37073.1 | 37073.1 |
| 5700 | 0.87 | 1.15% | 0.4342 | 434.2 | 377.7 |
| Surfactant | 0.95 | 0.50% | 0.1893 | 189.3 | 179.8 |
| NaOH sol. | 1.05 | 0.416% | 0.1576 | 157.6 | 165.5 |

### The volumes in columns 4 and 5 of TABLE I are in units of liters (L) and milliliters (mL), respectively, while the mass in column 6 is in units of grams (g). The bottom row lists the amounts for the 50 g/L NaOH solution previously described.

In use, the animal's ear canal may be filled with the ear treatment solution and massaged in, subsequently allowing the animal to shake the solution out. For ear treatment formulations which include an organosilane polymer antimicrobial ingredient such as, for example, AEM 5700 or similar product, it is believed that the organosilane polymer may also act as a bonding agent that attaches to the tissue within the ear canal and provides a mechanical barrier preventing the growth of bacteria within the ear canal, thus providing sustained protection. Additionally, if a holistic antiseptic is included, it is believed that such holistic antiseptic will be at least partially effective in killing bacteria that may lead to infection while treating the animal's ears. Weekly treatments should be sufficient for domestic canines and most other animals.

In accordance with another embodiment, a homeopathic animal ear treatment may include a homeopathic antimicrobial active ingredient, a bonding agent, and, optionally, one or more inactive ingredients such as for example, water and/or a holistic antiseptic.

Homeopathic antimicrobial agents which may be included in the homeopathic ear treatment include, for example, calendula officinalis tinc, mercurius solubilis, rhus toxicodendron or combinations thereof. Suitably, the homeopathic ear treatment formulation may contain up to about 30% by volume of a homeopathic antimicrobial ingredient or about 1% to about 30% by volume of a homeopathic antimicrobial agent. For example, a homeopathic ear treatment formulation according to the exemplary embodiment can include about 1% to about 10% by volume calendula officinalis tinc, about 1% to about 10% by volume mercurius solubilis and about 1% to about 10% by volume rhus toxicodendron.

The bonding agent may include an organosilane polymer such as, for example, 3-(trimethyloxysilyl) propyl octadecyl ammonium chloride or a blend of octadecylaminodimethyltrihydroxysilyl-propyl ammonium chloride and chloropropyltrihydroxysilane. Suitably, the homeopathic ear treatment formulation can include up to about 3% by volume bonding agent or about 0.25% to about 3% by volume bonding agent, or about 0.75% to about 1.25% by volume bonding agent.

Additionally, the homeopathic ear treatment formulation may include up to about 99% by volume inactive ingredients or about 60% to about 99% volume inactive ingredients. For example, the homeopathic ear treatment formulation may include about 60% to about 99% by volume water and/or about 0.5% to about 6% of a holistic antiseptic.

Table II below details an example of a homeopathic animal ear treatment according to this disclosure:

TABLE II

| | | | Percent |
|---|---|---|---|
| Ingredient | | | by volume |
| | Homeopathic | Calendula 45% Alcohol | 2.00% |
| | Active | Mercurius Solubilis H. 12X | 2.00% |
| | | (20% alcohol) | |
| | | Rhus toxicodendron 3X | 2.00% |
| | | (20% alcohol) | |
| | Bonding | 3-(trimethoxysilyl) propyl | 1.00% |
| | Agent | dimethyl octadecyl ammonium | |
| | | chloride | |
| | Inactive | Water | 91.50% |
| | | Eucalyptus oil | 0.75% |
| | | Tea tree oil | 0.75% |

Among the active ingredients listed above, calendula officinalis tinc and mercurius solubilis both serve as an anti-infective and/or anti-inflammatory, while rhus toxicodendron serves as an anti-itch and/or anti-inflammatory. The calendula officinalis tinc is preferably added in the form of a solution containing 45% by volume of alcohol, while the mercurius solubilis and the rhus toxicodendron are added in the form of a solution 20% by volume of alcohol. The active ingredients are micro-diluted in accordance with the Homeopathic Pharmacopoeia of the United States, and therefore should be non-toxic and cause no known side effects.

The inactive ingredients include one or more holistic antiseptics such as eucalyptus oil and/or tea tree oil, may be added to the ear treatment as desired. The 3-(trimethoxysilyl) propyl dimethyl octadecyl ammonium chloride serves as a preservative and/or bonding agent, which increases the duration of therapeutic benefits from application by five days or more. Other homeopathic active ingredients may be employed with the same or similar preservative and/or bonding agent. Many other homeopathic ingredients will work with similar benefits, provided the surfactant (the inactive preservative and/or bonding agent described above) is also employed.

The solution is preferably formulated by placing all of the required water in a container and, while stirring constantly, adding the inactive ingredients slowly to the water over the course of 5 minutes. An additional 5 minutes of stirring is allowed for the solution to mix thoroughly. Then, while stirring constantly, the required amounts of the active ingredients are slowly added over the course of another 5 minutes. Once the solution is complete, the solution should be shaken or otherwise agitated vigorously until completely mixed.

Prior to application, the solution should again be shaken vigorously. In application, the solution is applied by flushing out the ear canal of the animal and massaging the solution in to help clean the inner ear. After application, the animal will typically shake their head to remove excess fluid, and any resulting discharge should be wiped away. Treatment should include application once per week, or initially on two consecutive days and then once per week. The ear treatment described is suitable for the relief of chronic ear infection known as otitis externa and/or offensive otorrhea in dogs.

It should be noted that various substitutions may be made within the formulation of the present disclosure. For example, the Aegis Environments product “5772” contains the same compound as 5700, but in greater strength.

Although one or more embodiments are described herein in detail, those skilled in the art will understand that various changes, substitutions, variations, enhancements, nuances, gradations, lesser forms, alterations, revisions, improvements and knock-offs of the subject matter disclosed herein may be made without departing from the spirit and scope of the disclosure in its broadest form.

What is claimed is:

1. An animal ear treatment solution comprising:
an organosilane polymer antimicrobial in a concentration of approximately 0.25% to approximately 3.0% by weight; and
a surfactant.

2. The animal ear treatment solution according to claim 1, wherein the organosilane polymer antimicrobial I present in a concentration of approximately 1% to approximately 1.25% by weight.

3. The animal ear treatment solution according to claim 1, further comprising:
a holistic anitseptic.

4. The animal ear treatment solution according to claim 3, wherein the holistic antiseptic comprises one or both of green tea oil and eucalyptus oil.

5. The animal ear treatment solution according to claim 3, wherein the solution comprises about 0.5% to about 6% by volume of holistic antiseptic.

6. The animal ear treatment solution according to claim 3, wherein the solution comprises about 1% to about 2% by volume of holistic antiseptic.

7. The animal ear treatment solution according to claim 1, wherein the solution has a pH of at least 6 but not greater than 12.

8. The animal ear treatment solution according to claim 7, wherein the solution has a pH of approximately 10.

9. A method of treating an animal ear, comprising:
introducing a solution containing an organosilane polymer antimicrobial in a concentration of approximately 0.25% to approximately 3.0% by weight into an ear canal of an animal, wherein the organosilane polymer antimicrobial attaches to tissue within the ear canal to form a mechanical barrier preventing growth of bacteria on the tissue.

10. The method according to claim 9, wherein the solution contains a homeopathic active ingredient.

11. A homeopathic animal ear treatment solution comprising:
an organosilane polymer antimicrobial in a concentration of approximately 0.25% to approximately 3.0% by weight; and
a homeopathic active ingredient.

12. The animal ear treatment solution according to claim 11, further comprising:
about 0.25% to about 3% by volume bonding agent.

13. The animal ear treatment solution according to claim 12, further comprising:
about 0.75% to about 1.25% by volume bonding agent.

14. The animal ear treatment solution according to claim 11, further comprising:
a holistic anitseptic.

15. The animal ear treatment solution according to claim 14, further comprising:
about 60% to about 99% by volume water.

16. The animal ear treatment solution according to claim 14, further comprising:
about 0.5% to about 6% by volume of the holistic antiseptic.

17. The animal ear treatment solution according to claim 14, wherein the holistic antiseptic comprises one or both of tea tree oil and eucalyptus oil.

18. The animal ear treatment solution according to claim 11, further comprising:
about 1% to about 10% by volume calendula officinalis tinc;
about 1% to about 10% by volume mercurius solubilis; and
about 1% to about 10% by volume rhus toxicodendron.

19. The animal ear treatment solution according to claim 11, wherein the solution has a pH of greater than 6 but not greater than 12.

20. The animal ear treatment solution according to claim 19, wherein the solution has a pH of approximately 10.
